(19) United States
(12) Patent Application Publication
(10) Pub. No.: US 2018/0003479 A1
(43) Pub. Date: Jan. 4, 2018

(54) IMAGE PROCESSING APPARATUS AND IMAGE PROCESSING METHOD

(71) Applicant: CANON KABUSHIKI KAISHA, Tokyo (JP)

(72) Inventors: Nobuhiro Tomatsu, Yokohama-shi (JP); Toshiharu Sumiya, Hiratsuka-shi (JP); Makoto Sato, Tokyo (JP); Yoshihiko Iwase, Yokohama-shi (JP); Isao Komine, Sagamihara-shi (JP)

(21) Appl. No.: 15/541,912

(22) PCT Filed: Dec. 22, 2015
(86) PCT No.: PCT/JP2015/006376

§ 371 Date: Jul. 6, 2017

(30) Foreign Application Priority Data
Jan. 7, 2015 (JP) 2015-001678
Jan. 7, 2015 (JP) 2015-001679
Nov. 30, 2015 (JP) 2015-234268
Nov. 30, 2015 (JP) 2015-234269

(51) Int. Cl. G01B 9/02; G06T 7/11; G06T 7/00; G06K 9/46; A61B 3/00; A61B 3/10

(52) U.S. Cl. CPC G01B 9/02001; G01B 9/02092; A61B 3/0025; A61B 3/102; G06T 7/0012; G06K 9/4604; G06T 7/11; G01B 9/02089; G01B 9/02087; G01B 2290/70; G06T 2207/10101; G06T 2207/30041

(57) ABSTRACT

The present invention relates to accurately determining a contour of a depolarizing region.

An image processing apparatus extracts a depolarizing region in a polarization-sensitive tomographic image of a subject's eye, and detects, in a tomographic intensity image of the subject's eye, a region corresponding to the extracted depolarizing region. The tomographic intensity image corresponds to the polarization-sensitive tomographic image,

### TECHNICAL FIELD

The present invention relates to an image processing apparatus and an image processing method that process a polarization-sensitive tomographic image of a subject's eye.

### BACKGROUND ART

In recent years, optical coherence tomography (OCT) apparatuses using interference of low coherence light have been put to practical use. Such OCT apparatuses are capable of acquiring a tomographic image of a specimen with high resolution in a non-invasive manner. Therefore, particularly in the field of ophthalmology, OCT apparatuses are becoming indispensable for acquiring a tomomphic image of the fundus of a subject's eye. In fields other than ophthalmology, OCT apparatuses have been developed for tomographic observation of skin, or have been configured as endoscopes or catheters for capturing a tomographic image of the wall of a digestive or circulatory organ.

An ophthalmologic OCT apparatus has been developed to acquire not only a normal OCT image (also referred to as an intensity image) showing the shape of fundus tissue, but also a functional OCT image showing the optical characteristics and movement of fundus tissue. In particular, a polarization OCT apparatus capable of visualizing a nerve fiber layer and a retinal layer has been developed as a functional OCT apparatus, and its application to glaucoma and age-related macular degeneration has been studied. Techniques, using the polarization OCT apparatus, for detecting degeneration of a retinal layer and determining the progression of disease and the effect of disease treatment have also been studied.

The polarization OCT apparatus is capable of generating a polarization OCT image using a polarization parameter (retardation, orientation, or degree of polarization uniformity (DOPU)), which is an optical characteristic of fundus tissue, for identification and segmentation of the fundus tissue. Generally, the polarization OCT apparatus has an optical system configured to vary the polarization state of measuring light and reference light of the OCT apparatus by using a wave plate (e.g., λ/4 plate or λ/2 plate). The polarization OCT apparatus controls the polarization of light emitted from a light source, uses light modulated into a desired polarization state as measuring light for observing a sample, splits interference light into two orthogonal linearly polarized beams, detects them, and generates a polarization OCT image. NPL 1 discloses a method for specifically extracting, from a DOPU image reconstructed using DOPU parameters determined by threshold processing, a retinal pigment epithelium (RPE) layer, which is a depolarizing region (a region with depolarizing properties). The depolarization is a measure indicating the degree to which the polarization is eliminated in a subject to be examined. The depolarization is considered to be caused, for example, by random changes in the direction and phase of polarization resulting from reflection of measuring light in a micro-structure (e.g., melanin) in a tissue.

### CITATION LIST

### Non Patent Literature

NPL 1: Stefan Zotter et al, “Large-field high-speed polarization sensitive spectral domain OCT and its applications in ophthalmology” Biomedical Optics Express 3(11)

### SUMMARY OF INVENTION

### Solution to Problem

An image processing apparatus according to an aspect of the present invention includes an extracting unit configured to extract a depolarizing region in a polarization-sensitive tomographic image of a subject's eye; and a detecting unit configured to detect, in a tomographic intensity image of the subject's eye, a region corresponding to the extracted depolarizing region, the tomographic intensity image corresponding to the polarization-sensitive tomographic image.

An image processing apparatus according to another aspect of the present invention includes an extracting unit configured to extract a depolarizing region in a polarization-sensitive tomographic image of a subject's eye; a detecting unit configured to detect, in a tomographic intensity image of the subject's eye, a region corresponding to the extracted depolarizing region, the tomographic intensity image corresponding to the polarization-sensitive tomographic image; and a display control unit configured to cause a display unit to display the detected region over the tomographic intensity image.

An image processing apparatus according to another aspect of the present invention includes an extracting unit configured to extract a depolarizing region in a polarization-sensitive tomographic image of a subject's eye; a detecting unit configured to detect, in a tomographic intensity image of the subject's eye, a region corresponding to the extracted depolarizing region, the tomographic intensity image corresponding to the polarization-sensitive tomographic image; and a calculating unit configured to calculate a size of the detected region.

An image processing method according to another aspect of the present invention includes an extracting step of extracting a depolarizing region in a polarization-sensitive tomographic image of a subject's eye; and a detecting step of detecting, in a tomographic intensity image of the subject's eye, a region corresponding to the extracted depolarizing region, the tomographic intensity image corresponding to the polarization-sensitive tomographic image.

An image processing method according to another aspect of the present invention includes an extracting step of extracting a depolarizing region in a polarization-sensitive tomographic image of a subject's eye; a detecting step of detecting, in a tomographic intensity image of the subject's eye, a region corresponding to the extracted depolarizing region, the tomographic intensity image corresponding to the polarization-sensitive tomographic image; and a display step of causing a display unit to display the detected region over the tomographic intensity image.

An image processing method according to another aspect of the present invention includes an extracting step of extracting a depolarizing region in a polarization-sensitive tomographic image of a subject's eye; a detecting step of detecting, in a tomographic intensity image of the subject's eye, a region corresponding to the extracted depolarizing region, the tomographic intensity image corresponding to the polarization-sensitive tomographic image; and a calculating step of calculating a size of the detected region.

Further features of the present invention will become apparent from the following description of exemplary embodiments with reference to the attached drawings.

### BRIEF DESCRIPTION OF DRAWINGS

FIG. 1 is a schematic view illustrating an overall configuration of a polarization OCT apparatus according to a first embodiment.

FIG. 2A illustrates an image generated by a signal processing unit according to the first embodiment.

FIG. 2B illustrates another image generated by the signal processing unit according to the first embodiment.

FIG. 3 is a flowchart illustrating a processing operation in the polarization OCT apparatus according to the first embodiment.

FIG. 4A illustrates an intensity image containing hard exudates.

FIG. 4B illustrates a DOPU image containing the hard exudates.

FIG. 4C illustrates another DOPU image containing the hard exudates.

FIG. 4D is an enlarged view of a hard exudate region illustrated in FIG. 4A.

FIG. 5 is a diagram for explaining an image display screen according to the first embodiment.

FIG. 6 is a flowchart illustrating a process of image analysis according to a second embodiment.

FIG. 7A illustrates a two-dimensional image generated according to the second embodiment.

FIG. 7B also illustrates the two-dimensional image generated according to the second embodiment.

FIG. 8 shows a list of regions, each identified as a geographic atrophy, according to the second embodiment.

### DESCRIPTION OF EMBODIMENTS

A DOPU image is generally obtained by calculating, for each region, a DOPU parameter determined using tomographic image data acquired by a polarization OCT apparatus, and two-dimensionally reconstructing the resulting DOPU parameters. A DOPU parameter is a parameter representing the degree of polarization of light and taking on values from 0 to 1. The DOPU parameter takes on a value of 1 if detected light is completely polarized, and takes on a value of 0 if detected light is not polarized and the polarization state is non-uniform. The degree of polarization of a depolarizing region (a region with depolarizing properties) is lower than that of return light from other tissues. The DOPU parameter can he calculated for each pixel. However, since polarization states in a given range of space including the pixel are statistically processed (i.e., their average value is determined), the resolution of the resulting DOPU image is lower than that of a normal intensity image. This makes it difficult to accurately capture the contour (range) of a depolarizing region. Accordingly, the present embodiment provides a technique for accurately determining the contour (range) of a depolarizing region.

An image processing apparatus according to the present embodiment includes an extracting unit configured to extract a depolarizing region (a region with depolarizing properties) in a polarization-sensitive tomographic image (e.g., DOPU image) of a subject's eye. The extracting unit may directly extract a depolarizing region from the polarization-sensitive tomographic image, or may extract a signal corresponding to the depolarizing region from signals present before generation of the polarization-sensitive tomographic image. The depolarizing region is a region containing, for example, an RPE layer or hard exudates. The image processing apparatus of the present embodiment also includes a detecting unit configured to detect, in a tomographic intensity image of the subject's eye, a region corresponding to the extracted depolarizing region. The tomographic intensity image corresponds to the polarization-sensitive tomographic image. The detecting unit may directly detect the region from the tomographic intensity image, or may detect a signal corresponding to the region from signals present before generation of the tomographic intensity image. The contour (range) of the depolarizing region can thus be accurately determined.

The image processing apparatus of the present embodiment may include a display control unit configured to cause a display unit to display the detected region over the tomographic intensity image. The depolarizing region can thus be accurately displayed,

The image processing apparatus of the present embodiment may include a calculating unit configured to calculate a size of the detected region. The size of the detected region may be a volume if the polarization-sensitive tomographic image is a three-dimensional image. If the polarization-sensitive tomographic image is a two-dimensional image, the size of the detected region may be an area. Even when the polarization-sensitive tomographic image is a three-dimensional image, an area may be calculated as the size of the detected region. Besides the volume and area, the size of the detected region may be a width or perimeter. The size of the depolarizing region can thus be accurately determined,

By using a DOPU image, hard exudates in a patient with diabetic retinopathy may be extracted as a depolarizing region. A hard exudates region is a degenerated region with depolarizing properties, and the relationship between hard exudates and the progression of disease in patients with diabetic retinopathy has been studied. In the image processing apparatus of the present embodiment, the depolarizing region may be a hard exudate region. In this case, the contours of hard exudates can be accurately determined, and the hard exudates can be accurately displayed. Thus, in a follow-up, such as monitoring of the progression and treatment of hard exudates, the user can easily identify changes in the size and number of hard exudates while viewing the hard exudates displayed on a monitor. Also, the size of the hard exudates can be accurately, determined. This allows quantitative assessment of changes in the size and number of hard exudates in a follow-up, such as monitoring of the progression and treatment of hard exudates,

### First Embodiment: Accurate Detection of Contour of Hard Exudate Region

An embodiment of the present invention will now be described in detail with reference to the drawings.

### Overall Configuration of Apparatus

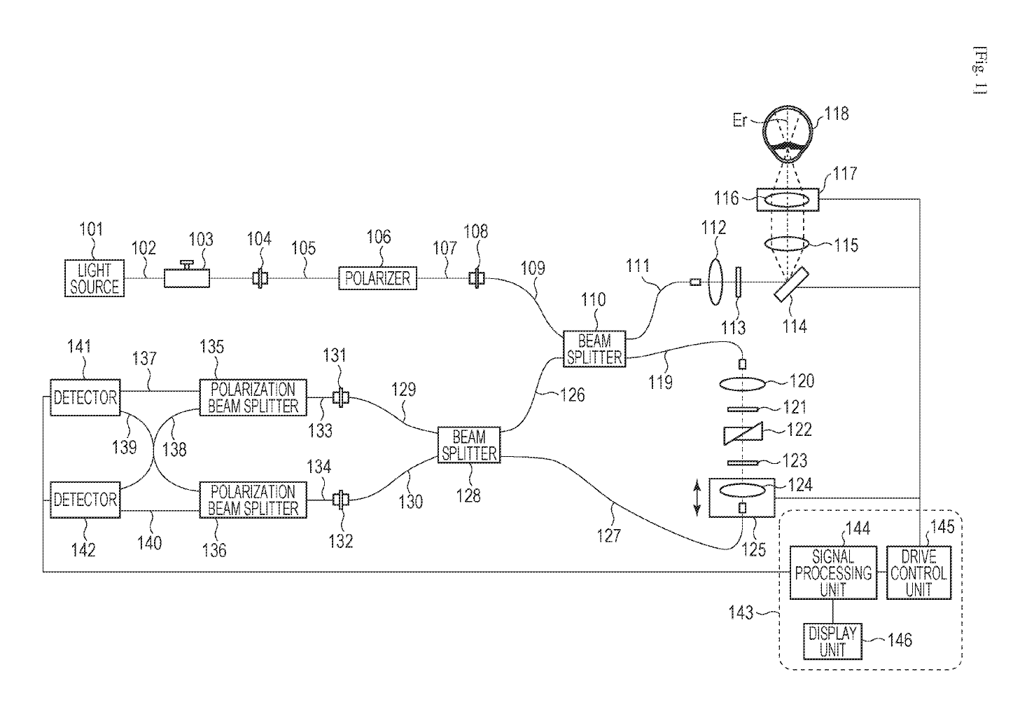

FIG. 1 is a schematic view illustrating an overall configuration of a polarization OCT apparatus which is a tomographic imaging apparatus according to the present embodiment. A polarization OCT apparatus based on swept source OCT (SS-OCT) will be described in the present embodiment. Note that the present invention is not limited to this, and is also applicable to a polarization OCT apparatus based on spectral domain OCT (SD-OCT).

### Configuration of Polarization OCT Apparatus 100

A configuration of a polarization OCT apparatus 100 will be described. A light source 101 is a swept source (SS) light source that emits light while sweeping the wavelength centered at 1050 nm with a sweep width of 100 nm. The light emitted from the light source 101 is guided through a single mode fiber (SM fiber) 102, a polarization controller 103, a connector 104, an SM fiber 105, a polarizer 106, a polarization maintaining fiber (PM fiber) 107, a connector 108, and a PM fiber 109 to a beam splitter 110, by which the light is split into measuring light (which may also be referred to as OCT measuring light) and reference light (which may also be referred to as reference light corresponding to OCT measuring light). The splitting ratio between the reference light and the measuring light, which are obtained by the beam splitter 110, is 90:10. The polarization controller 103 is capable of changing the polarization of light emitted from the light source 101 into a desired polarization state. The polarizer 106 is an optical element having a characteristic of allowing transmission of only a specific linearly polarized component. Generally, most of the light emitted from the light source 101 has a high degree of polarization and is polarized in a specific direction, but the light includes a component called a randomly polarized component having no specific polarization direction. The randomly polarized component is known to degrade the quality of a polarization OCT image, and thus is cut by the polarizer 106. Since only specific light in a linearly polarized state can pass through the polarizer 106, the polarization controller 103 adjusts the polarization state to allow a desired amount of light to enter a subject's eye 118.

The measuring light from the beam splitter 110 passes through a PM fiber 111 and is collimated by a collimator 112. The collimated measuring light passes through a quarter-wave plate 113 and further passes through a galvano scanner 114 for scanning a fundus Er of the subject's eye 118 with the measuring light, a scan lens 115, and then through a focus lens 116 to enter the subject's eye 118. The galvano scanner 114, which has been described as a single mirror, is actually formed by two galvano scanners for raster-scanning the fundus Er of the subject's eye 118. The galvano scanner 114 may be firmed by a single mirror capable of scanning with light in a two-dimensional direction. The two galvano scanners described above may be arranged close to each other, or positioned to be optically conjugate with the front portion of the subject's eye 118. The focus lens 116 is secured onto a stage 117. The focus of the focus lens 116 can be adjusted by moving the stage 117 in the optical axis direction. The galvano scanner 114 and the stage 117 are controlled by a drive control unit 145, so that the fundus Er of the subject's eye 118 can be scarified with the measuring light in a desired range (e.g., acquisition range or position of a tomographic: image, or irradiation position of measuring light). The quarter-wave plate 113 is an optical element having a characteristic of delaying, by a quarter wavelength, the phase between the optical axis of a quarter-wave plate and an axis orthogonal to the optical axis. In the present embodiment, with respect to the direction of linear polarization of the measuring light from the PM fiber 111, the quarter-wave plate 113 is rotated by 45° about its optical axis to produce circularly polarized light, which enters the subject's eye 118.

Although no detailed description is given in the present embodiment, the method of the present embodiment is also applicable to the case of having a tracking function which detects the movement of the fundus Er and scans the fundus Er by causing the mirror of the galvano scanner 114 to follow the movement of the fundus Er. The tracking can he done using a commonly used technique, either on a real time basis or by post-processing. For example, the tracking can be done, using a scanning laser ophthalmoscope (SLO). In this technique, after a two-dimensional image of the fundus Er in a plane perpendicular to the optical axis is acquired over time using the SLO, a feature portion, such as a vascular bifurcation, in the image is extracted. Then, how the feature portion in the acquired two-dimensional image has moved is calculated as the amount of movement of the fundus Er. Thus, real-time tracking can be done by feeding the calculated amount of movement back to the galvano scanner 114.

The measuring light enters the subject's eye 118 through the focus lens 116 on the stage 117, and is focused onto the fundus Er. After irradiation of the fundus Er, the measuring light is reflected and scattered by each retinal layer and returned through the above-described optical path to the beam splitter 110. From the beam splitter 110, the returned measuring light passes through a PM fiber 126 and enters a beam splitter 128.

On the other hand, the reference light from the beam splitter 110 passes through a PM fiber 119 and is collimated by a collimator 120. The collimated reference light passes through a half-wave plate 121, a dispersion compensation glass 122, a neutral density (ND) filter 123, and a collimator 124 to enter a PM fiber 127. The collimator 124 and an end of the PM fiber 127 are secured onto a coherence gate stage 125, and controlled by the drive control unit 145 to be driven in the optical axis direction in accordance with the axial length of the subject's eye 118. The half-wave plate 121 is an optical element having a characteristic of delaying, by a half wavelength, the phase between the optical axis of a half-wave plate and an axis orthogonal to the optical axis. In the present embodiment, an adjustment is made such that the long axis of linearly polarized reference light from the PM fiber 119 is tilted by 45° in the PM fiber 127. Although the optical path length of the reference light is changed in the present embodiment, it is only necessary that the difference in length between the optical paths of the measuring light and the reference light be changed,

The reference light passed through the PM fiber 127 enters the beam splitter 128, by, which the returned reference light and the reference light are multiplexed into interference light and then split into two. The resulting interference beams (i.e., positive and negative components of the interference light) have opposite phases. The positive component of the interference light passes through a PM fiber 129, a connector 131, and a PM fiber 133 to enter a polarization beam splitter 135. The negative component of the interference light passes through a PM fiber 130, a connector 132, and a PM fiber 134 to enter a polarization beam splitter 136,

The polarization beam splitters 135 and 136 each split the interference light, in accordance with two orthogonal polarization axes, into two light beams, a vertical polarization component (hereinafter referred to as V polarization component) and a horizontal polarization component (hereinafter referred to as H polarization component). The positive component of the interference light that has entered the polarization beam splitter 135 is split by the polarization beam splitter 135 into two interference light beams, a positive V polarization component and a positive H polarization component. The positive V polarization component passes through a PM fiber 137 to enter a detector 141, whereas the positive H polarization component passes through a PM fiber 138 to enter a detector 142. On the other hand, the negative component of the interference light that has entered the polarization beam splitter 136 is split by the polarization beam splitter 136 into a negative V polarization component and a negative H polarization component. The negative V polarization component passes through a PM fiber 139 to enter the detector 141, whereas the negative H polarization component passes through a PM fiber 140 to enter the detector 142.

The detectors 141 and 142 are both differential detectors. When two interference signals with a phase difference of 180° are input, the detectors 141 and 142 each remove a direct-current component and output only an interference component.

The V polarization component of the interference signal detected by the detector 141 and the H polarization component of the interference signal detected by the detector 142 are each output as an electric signal corresponding to the intensity of light and input to a signal processing unit 144 serving as a tomographic; image generating unit.

### Controller 143

A controller 143, which is an example of the image processing apparatus of the present embodiment, will be described. The controller 143 is connected to the tomographic imaging apparatus of the present embodiment to be able to communicate therewith. The controller 143 may be either integral with or separate from the tomographic imaging apparatus. The controller 143 includes the signal processing unit 144, the drive control unit 145, and a display unit 146. The drive control unit 145 controls each part as described above. On the basis of the signals output from the detectors 141 and 142, the signal processing unit 144 generates an image, analyzes the generated image, and generates visualized information. representing the analysis result. That is, the signal processing unit 144 serves as a display control unit capable of causing the display unit 146 to display, on its display screen, the generated image and the analysis result described above. The display control unit may be provided separately from the signal processing unit 144. The display unit 146 is, for example, a liquid crystal display. The image data generated by the signal processing unit 144 may be transmitted to the display unit 146 through either wired or wireless communication. Although the display unit 146 is included in the controller 143 in the present embodiment, the present invention is not limited to this, and the display unit 146 may be separate from the controller 143. For example, the display unit 146 may be provided as a tablet, which is a user-portable device. In this case, the display unit 146 may have a touch panel function which allows the user to move the display position of the image, scale the image, and change the displayed image on the touch panel.

### Image Processing

Image generation in the signal processing unit 144 will now be described. The signal processing unit 144 performs general reconstruction processing on the interference signals output from the detectors 141 and 142 to generate two tomographic images based on the respective polarization components, a tomographic image corresponding to the V polarization component and a tomographic image corresponding to the H polarization component.

First, the signal processing unit 144 removes fixed pattern noise from the interference signals. This is done by extracting fixed pattern noise by averaging a plurality of detected A-scan signals, and subtracting the extracted fixed pattern noise from the input interference signals. Next, the signal processing unit 144 performs windowing to optimize a depth resolution and a dynamic range which have a trade-off relationship when the Fourier transform is performed over a finite interval. Cosine taper windowing is performed in the present embodiment. Then, the signal processing unit 144 performs fast Fourier transform (FFT) processing to generate tomographic signals. By performing the above-described processing on the interference signals of two polarization components, two tomographic images are generated. The windowing method is not limited to cosine taper windowing, and the operator may select any method appropriate for the purpose. Generally known windowing, such as Gaussian windowing or harming windowing, is also applicable here.

Generation of Intensity image (Tomographic intensity image)

The signal processing unit 144 generates an intensity image from the two tomographic signals described above. The intensity image is basically the same as a tomographic image in the OCT of the related art, and may also be referred to as a tomographic intensity image in the present specification. A pixel value r in the tomographic intensity image is calculated by Equation 1 using an amplitude Aᵥ of the V polarization component and an amplitude A_{H} of the H polarization component obtained by the detectors 141 and 142.

### [Math. 1]

r=\sqrt{A_{H}²+A_{V}²} Equation 1

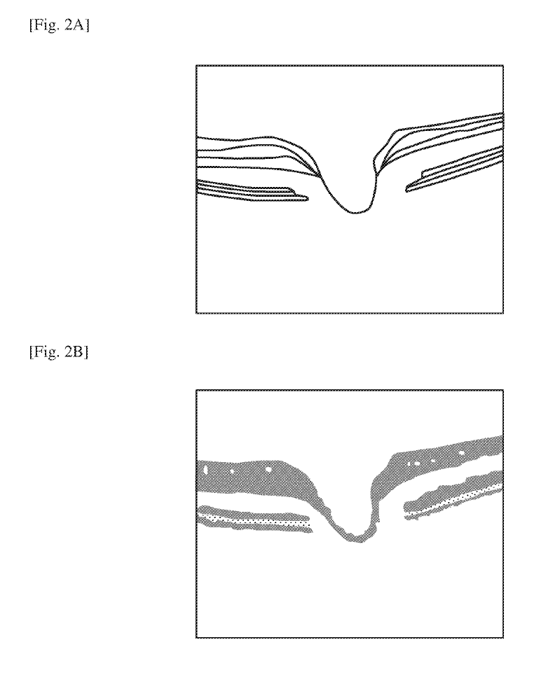

FIG. 2A illustrates an intensity image of an optic disk portion. The gal vano scanner 114 raster-scans the fundus Er of the subject's eye 118 to obtain a B-scan image of the fundus Er. By acquiring a plurality of B-scan images at different positions on the fundus Er in the sub-scanning direction, volume data of the intensity image is generated.

### Generation of DOPU Image

The signal processing unit 144 calculates a Stokes vector S for each pixel from Equation 2 using the acquired amplitudes A_{H} and A_{V} and a phase difference ΔΦ therebetween:

$$\begin{matrix}[ Math . 2 ] & \\ S = ( \begin{matrix}I \\ Q \\ U \\ V\end{matrix} ) = ( \begin{matrix}{A}_{H}^{2} + {A}_{V}^{2} \\ {A}_{H}^{2} + {A}_{V}^{2} \\ 2 {A}_{H} {A}_{V} cos Δ φ \\ 2 {A}_{H} {A}_{V} sin Δ φ\end{matrix} ) & Equation 2\end{matrix}$$

where the phase difference ΔΦ is calculated as ΔΦ=Φ_{V}−Φ_{H} using phases Φ_{H} and Φ_{V} of the respective signals obtained in the calculation of two tomographic images.

Next, the signal processing unit 144 sets windows with a size of about 70 μm in the main scanning direction of the measuring light and about 1 μm in the depth direction thereof for each B-scan image, averages elements of Stokes vectors S calculated for respective pixels by Equation 2 in each window, and calculates the degree of polarization uniformity (DOPU) in the window from Equation 3:

### [Math.3]

DOPU=\sqrt{Qₘ²+Uₘ²+Vₘ²} Equation 3

where Qₘ, Uₘ, and Vₘ, are values obtained by averaging elements Q, U, and V Of Stokes vector S in each window.

By performing the above-described processing for all windows in the B-scan image, a DOPU image (also referred to as a tomographic image representing the degree of polarization uniformity) of the optic disk portion illustrated in FIG. 2B is generated.

DOPU is a numerical value representing the degree of polarization uniformity. The DOPU is close to 1 in an area where polarization is maintained, and is less than 1 in an area where polarization is eliminated and not maintained. In a structure in the retina, the RPE layer has depolarizing properties. Therefore, in the DOPU image, a portion corresponding to the RPE layer has a smaller DOPU than other areas. In FIG. 2B, a light-colored area represents an RPE layer, and a dark-colored area represents a retinal layer region where polarization is maintained. A depolarizing layer, such as the RPE layer, is visualized in the DOPU image. Therefore, even when the RPE layer is deformed by disease or the like, the RPE layer can be visualized more reliably than in the case of using variation in intensity. As in the intensity image, volume data of the DOPU image can be generated by arranging the acquired B-scan images in the subscanning direction. In the present specification, a DOPU image and a retardation image may also be referred to as a polarization-sensitive tomographic image. Also in the present specification, a DOPU image may also be referred to as an image showing depolarizing properties. Also in the present specification, a retardation map and a birefringent map generated from volume data of the retardation image may also be referred to as a polarization fundus image.

### Processing Operation

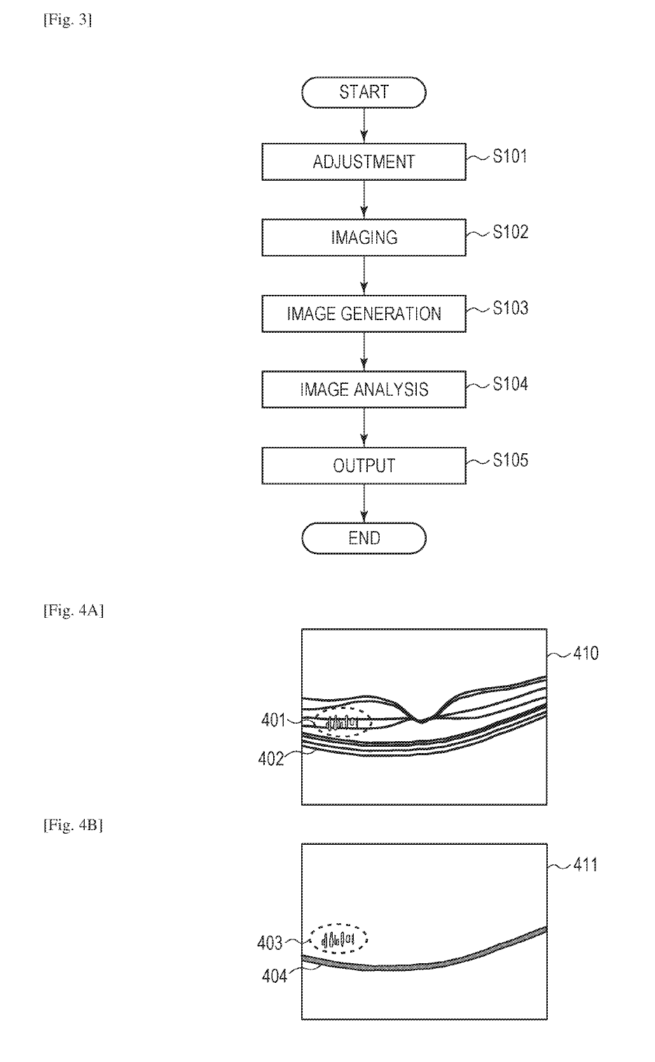

A processing operation in the polarization OCT apparatus of the present embodiment will now be described. FIG. 3 is a flowchart illustrating the processing operation in the polarization OCT apparatus.

### Adjustment

In step S101, with the subject's eye 118 placed on the polarization OCT apparatus, alignment of the polarization OCT apparatus and the subject's eye 118 is performed. Alignment of working distance or the like in the XYZ directions and adjustment of focus and coherence gate will not be described here, as they are done by techniques commonly used.

### Imaging and Image Generation

In steps S102 and S103, light emitted from the light source 101 is split into measuring light and reference light. Interference light of return light (which is measuring light reflected or scattered by the fundus Er of the subject's eye 118) and the reference light is received by the detectors 141 and 142, and the signal processing unit 144 generates each image as described above.

### Analysis

Detection of Hard Exudates in DOPU Image

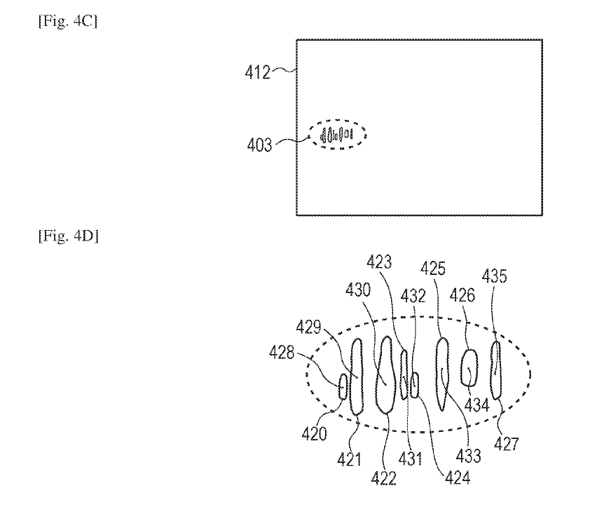

In step S104, the signal processing unit 144 detects hard exudates in the generated DOPU image. FIG. 4A illustrates an intensity image 410 containing hard exudates, and FIGS. 4B and 4C illustrate DOPU images 411 and 412, which visualize depolarizing properties of a substance to be measured. The intensity image 410 illustrated in FIG. 4A visualizes not only a hard exudate region 401 and an RPE layer 402 having depolarizing properties, but also layers forming the retina. In contrast, the DOM image 411 illustrated in FIG. 4B visualizes only regions with depolarizing properties. In the present embodiment, a threshold for the DOPU of a region visualized in a DOPU image is 0.75, If the level of depolarizing properties of a region is higher, that is, if the DOPU of a region where the degree of polarization of light returned by reflection or scattering is low is less than 0.75 (DOP(<0.75), the region is visualized in the DOPU image 411. A hard exudate region 403 and a RPE layer 404 are thus visualized in the DOPU image 411. Although the threshold for the DOM is 0.75 in the present embodiment, the threshold is not limited to this, and can be set by the examiner depending on the object to he measured and the purpose of the measurement.

In the present embodiment, the signal processing unit 144 identifies the RPE layer 404 in the DOPU image 411, and removes the identified. RPE layer 404 from the regions with depolarizing properties to extract the hard exudate region 403. The extraction can be done by using the fact that the hard exudate region 403 is on the inner layer side of the RPE layer 404, or by using the geometrical feature of the hard exudate region 403 of having no continuous layer structure. For example, the signal processing unit 144 may calculate the coordinates of the RPE layer 402 by performing segmentation of layers using the intensity image 410, and remove DOPU data near the calculated coordinates in the DOPU image 411. Alternatively, the signal processing unit 144 may extract a region with a high DOPU density from the DOPU image 411 using a graph-cut technique, and remove DOPU data near a line obtained by fitting. By, performing such processing, the hard exudate region 403 can be specifically extracted in the DOPU image 412 (see FIG. 4C). By performing the above-described processing on all B-scan images forming volume data of the acquired DOPU image, the signal processing unit 144 specifically extracts a hard exudate region in the volume data.

Identification of Hard Exudate Positions in intensity image Using Hard Exudates Detected in DOPU Image

After specifically extracting the hard exudate region 403, the signal processing unit 144 acquires the coordinate values of the hard exudate region 403 from the DOPU image 412. As described above, a DOM image is generated by determining the Stokes vector S for each pixel from acquired amplitudes A_{H} and A_{V} and a phase difference ΔΦ therebetween, and averaging elements of the resulting Stokes vectors S to obtain DOPU in a B-scan image. Therefore, the image size and the pixel pitch are unchanged. That is, the DOPU image and the tomographic intensity image are positionally associated with each other. The DOPU image and the tomographic intensity image may be acquired at different time points, or by different optical systems. In this case, these images can be made positionally associated with each other by performing alignment therebetween using image correlation or the like. Therefore, by applying the coordinate values acquired in the DOPU image 412 to the intensity image 410, the position of the hard exudate region 403 in the DOPU image 412 can be identified in the intensity image 410,

FIG. 4D is an enlarged view of the hard exudate region 401. The hard exudate region 403 contains DOPU images corresponding to hard exudates 420 to 427. The signal processing unit 144 calculates the coordinates of each hard exudate. It is not essential here to obtain coordinate information of the entire area of each hard exudate, and it is only necessary to include part of each hard exudate. For example, the coordinate values acquired in the DOPU image 412 may be the values of barycentric coordinate points 428 to 435 of the extracted hard exudates 420 to 427, or the coordinates of leftmost pixels in the respective hard exudates 420 to 427 in the DOPU image 412. By performing this processing on all B-scan images forming the volume data of the acquired DOPU image 412, the coordinates of the hard exudates 420 to 427 within the volume data of the intensity image 410 can be identified.

Specific Detection of Hard Exudates in Intensity image

After identifying the coordinates of the hard exudates 420 to 427 in the intensity image 410, the signal processing unit 144 specifically extracts the hard exudates 420 to 427. Although a region growing method is used for the extraction in the present embodiment, the present invention is not limited to this. Any algorithm that performs region segmentation on the basis of a spatial initial position can be applied by determining the initial position in the DOPU image 412. For the coordinate values identified for each of the hard exudates 420 to 427, the signal processing unit 144 sets a seed point, and performs region growing using a threshold for the intensity image 410 as a criterion. That is, the signal processing unit 144 starts region growing in the intensity image 410 at the seed point determined in the DOPU image 412, and continues to perform the growing processing until the intensity value falls below threshold. Although the threshold can be experimentally determined, a condition may be added such that the range of growing does not exceed the range of the hard exudates 420 to 427 visualized in the DOPU image 412. The area defined by the contours of the hard exudates 420 to 427 identified in the DOPU image 412 may be larger than the actual hard exudates 420 to 427 due to the effect of window processing necessary for calculation of DOPU parameters. However, since the above-described processing determines the contours on the basis of the intensity image 410, the shapes of the hard exudates 420 to 427 can be accurately extracted. The processing described above can he performed on all B-scan images forming the volume data of the acquired intensity image 410, so that a hard exudate region in the volume data of the intensity image 410 can be identified. The present invention is also applicable to processing on only one B-scan image.

Display of Hard Exudates in Intensity image

After extraction of the hard exudates 420 to 427 described above, an image can be displayed by the display unit 146 in step S105. The hard exudates 420 to 427 in the intensity image 410, identified in step S104, are displayed over the intensity image 410 in an identifiable state. For example, for easy distinction of the hard exudates 420 to 427 in the intensity image 410 from other regions in the intensity image 410, the hard exudates 420 to 427 are displayed over the intensity image 410 in a color not used in the intensity image 410 (e.g., in red or yellow),

By using the tomographic imaging apparatus and the image processing method described above, a lesion area with depolarizing properties can be specifically displayed. Also, the size of the lesion area can be accurately displayed. Although the tomographic imaging apparatus of the present embodiment is formed only by the polarization OCT apparatus, combining a fundus observing apparatus, such as a scanning laser ophthalmoscope (SLO), with the polarization OCT apparatus and establishing a correspondence with the imaging position of the polarization OCT apparatus can provide more accurate diagnosis. Although the present embodiment deals with hard exudates, the present invention is not limited to this. The image processing method described above is applicable to display of any lesion that occurs in the fundus and has depolarizing properties. Although the present embodiment describes an image display method for only B-scan images of the polarization OCT apparatus, the present invention is not limited to this. For example, by acquiring three-dimensional data through multiple B-scans and performing the above-described image analysis on each of the B-scan images to generate volume data, the polarization OCT apparatus can three-dimensionally visualize a lesion area with depolarizing properties.

Calculation of Hard Exudate Region in Intensity image

After identifying a hard exudate region for all B-scan images forming the volume data of the intensity image as described above, the signal processing unit 144 may calculate the volume of the hard exudate region in the volume data. First, the signal processing unit 144 arranges all the acquired B-scan images of the intensity image in the sub-scanning direction (y-direction) in the order of acquisition to generate volume data of the intensity image. Next, for the hard exudate region identified for each of the B-scan images, the signal processing unit 144 extracts and combines pixels successively arranged, or partially in contact with each other, in the sub-scanning direction of each B-scan. The extraction is done using a region growing method, as in the extraction of hard exudates in a B-scan image. Last, the signal processing unit 144 calculates the volume of a voxel of extracted hard exudates by taking into account the pixel resolution for each of the axes of length (y-direction), width (x-direction), and depth (z-direction) of the volume data. In the present embodiment, a volume of 6 mm long, 8 mm wide, and 2 mm deep is imaged with a resolution of 256 pixels for the length, 512 pixels for the width, and 1024 pixels for the depth. Accordingly, dimensions for each pixel are 23 μm long, 16 μm wide, and 2 μm deep. These values are calculated for each of the hard exudates contained in the volume data.

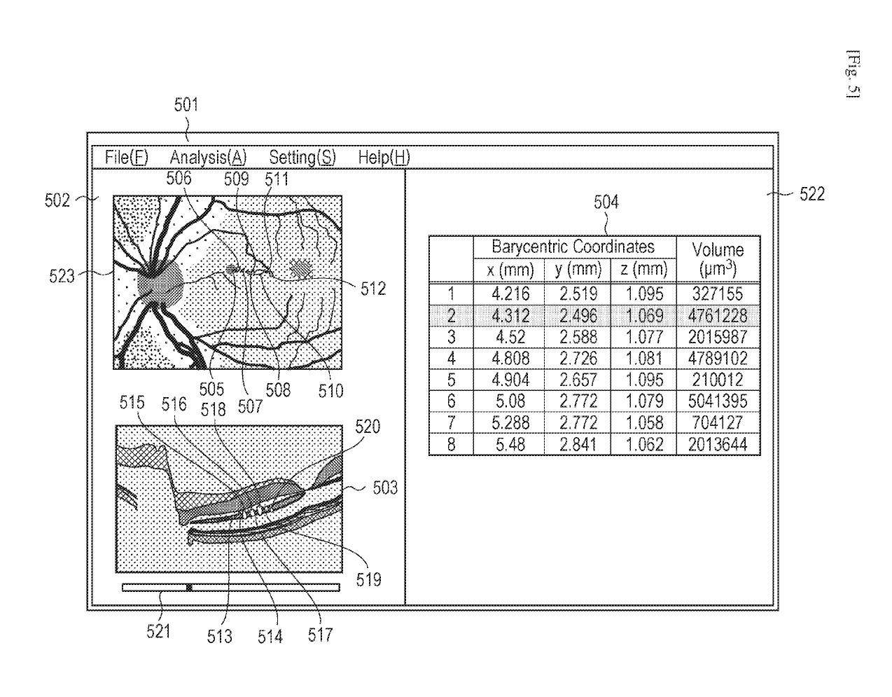

After the volume of each of the hard exudates is calculated as described above, a list of the volume values corresponding to the respective extracted hard exudates is displayed in the display unit 146. A display screen displayed by the display unit 146 is illustrated in FIG. 5. A display screen 501 contains an image display section 502 and a list display section 522. The image display section 502 shows an intensity image map 523 and a B-scan image 503 of the intensity image in an xy plane obtained from the generated volume data. Any of the acquired B-scan images can be displayed by moving a slider 521. The list display section 522 shows a list 504, which associates coordinate values and a volume value of each of the extracted hard exudates.

When the operator selects a row in the list 504, the corresponding one of hard exudate regions 505 to 512 and 513 to 520 in the intensity image map 523 and B-scan image 503 is highlighted. Conversely, when the operator selects one of the hard exudate regions 505 to 512 and 513 to 520 shown in the intensity image map 523 and B-scan image 503, the corresponding row in the list 504 is highlighted.

Although volume values are calculated for respective hard exudates in the present embodiment, the volume values of hard exudates present within any range may be summed and displayed. Although an intensity image map and a B-scan image of the intensity image are displayed in the present embodiment, the present invention is not limited to this. Any image may be displayed, which is selected from all images (including an En face map (En face image) and a DOPU image (DOPU map) obtained after segmentation) that can be acquired or generated by the polarization OCT apparatus. Note that the En face map is a two-dimensional image (projection image) obtained by projecting a predetermined three-dimensional range onto a predetermined plane. The predetermined plane is, for example, an xy plane, where Z=0. For example, the signal processing unit 144 (generating unit) can generate the two-dimensional image (projection image) of the predetermined range by summing intensities in the predetermined range in the depth direction. Any range in the depth direction may be selected as the predetermined range by using information at the boundary of layers obtained by segmentation. Also, the signal processing unit 144 can generate the two-dimensional image of the predetermined range by using a representative value, such as an average value, a central value, or a maximum value, of the intensities in the predetermined range in the depth direction. The two-dimensional image of the predetermined range may be generated by various known techniques.

By using the tomographic imaging apparatus and the image processing method described above, the volumes of hard exudates can be accurately calculated. As described in the present embodiment, by combining a fundus observing apparatus, such as a scanning laser ophthalmoscope (SLO), with the polarization OCT apparatus and establishing a correspondence with the imaging position of the polarization OCT apparatus, the calculation can be done more accurately. For example, by tracking the movement of the subject's eye on the basis of a fundus image acquired by the SLO and generating volume data by correcting the amount of movement of the subject's eye, it is possible to eliminate displacement of each B-scan caused by the movement of the subject's eye, and to accurately calculate the areas and volumes of hard exudates. Although the present embodiment deals with hard exudates, the present invention is not limited to this. The image processing method described above is applicable to calculation of the area and volume of any lesion that occurs in the fundus and has depolarizing properties. Although the present embodiment describes a method for calculating the volumes of hard exudates using volume data in the polarization OCT apparatus, the, present invention is not limited to this. For example, it is also possible to calculate the area of a lesion portion with depolarizing properties using a B-scan image, or to calculate the area of a lesion portion with depolarizing properties in an En face image.

### Second Embodiment: Accurate Detection of Range of Geographic Atrophy

The first embodiment describes a method for detecting hard exudates in a patient with diabetic retinopathy using a DOPU image. The present embodiment will describe an example of detecting a geographic atrophy (GA), which is a lesion associated with atrophic age-related macular degeneration. The geographic atrophy is a lesion where an atrophic region in an RPE layer with depolarizing properties is spread in a geographic pattern. The atrophic age-related macular degeneration is accompanied by this lesion. By detecting (extracting) a depolarizing region (i.e., region with depolarizing properties), the boundary of atrophy in the RPE layer becomes clearly visible if there is geographic atrophy.

In an image processing apparatus according to the present embodiment, by detecting (identifying) a discrete region in an RPE layer with depolarizing properties in a DOPU image, the discrete region can be accurately displayed and analyzed as geographic atrophy in an En face map (En face image) of the RPE layer obtained after segmentation. Thus, in a follow-up, such as monitoring of the progression and treatment of atrophic age-related macular degeneration, the user can easily identify changes in the size and number of geographic atrophies while viewing the analysis of the geographic atrophies displayed on a monitor. Also, the size of the geographic atrophies can be accurately determined. This allows quantitative assessment of changes in the size and number of geographic atrophies in a follow-up, such as monitoring of the progression and treatment of atrophic age-related macular degeneration. Note that the En face map of the RPE layer is a two-dimensional image (projection image) obtained by projecting a three-dimensional RPE layer onto a predetermined plane. The predetermined plane is, for example, an xy plane, where Z=0. For example, the signal processing unit 144 (generating unit) can generate the two-dimensional image (projection image) of the RPE layer by summing intensities in the RPE layer in the depth direction. Also, the signal processing unit 144 can generate the two-dimensional image of the RPE layer by using a representative value, such as an average value, a central value, or a maximum value, of the intensities in the RPE layer in the depth direction. The two-dimensional image of the RPE layer may be generated by various known techniques.

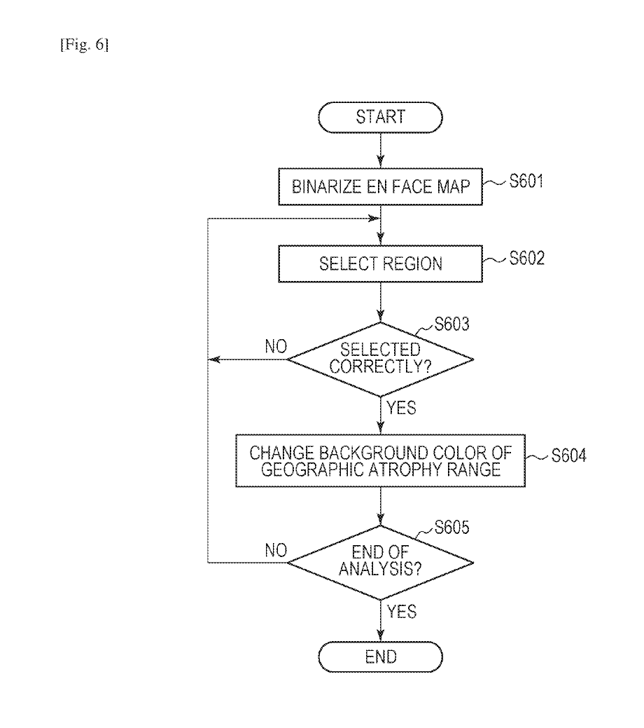

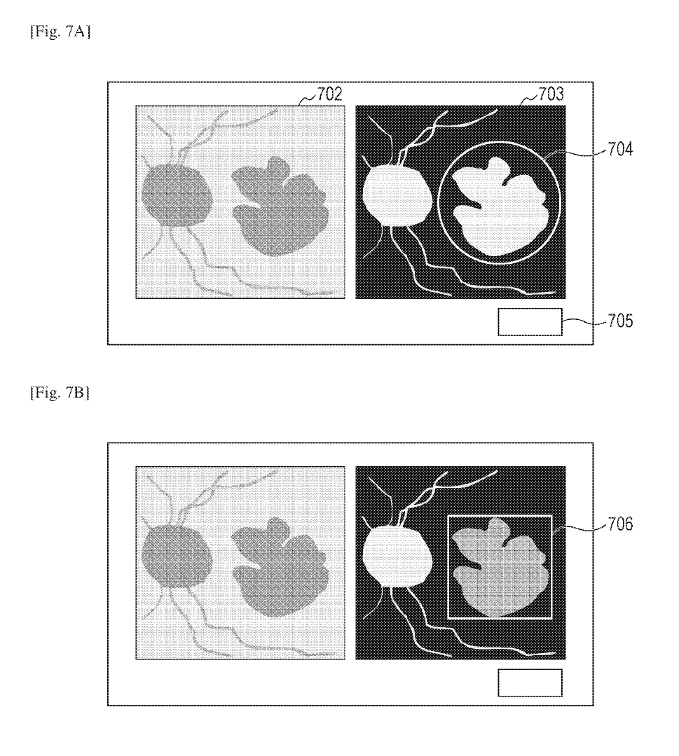

The configuration of the apparatus and the image forming method of the present embodiment are the same as those of the first embodiment, and thus will not be described here. The differences from the first embodiment are step S104 and step S105 of FIG. 3, and they will now be described. The image analysis of step S104 in the present embodiment will be described in accordance with the processing flow of FIG. 6, in substantially the same manner as the first embodiment, threshold processing is performed on the DOPU image generated in step S103 of FIG. 3, whereby an RPE layer can be detected (extracted) as a depolarizing region at any depth position. FIG. 7A illustrates an En face map 702 of the detected RPE layer generated by the signal processing unit 144 (generating unit) through the use of coordinates of the RPE layer in the depth direction. Discrete regions, such as an optic disk, blood vessels, and a defect in the RPE layer, can be viewed on the En face map 702. Although the En face map 702 of the RPE layer is used in the present embodiment, the image to be used is not limited to this. For example, a map showing a layer structure including the RPE layer, obtained by segmentation in the range of 2.0 μm above a choroid, may be used.

In the image analysis of the present embodiment, first, in step S601 of FIG. 6, the signal processing unit 144 binarizes the En face map 702 of the RPE layer to generate a binary map 703. The binarization can be done by correcting, after binarizing an En face map based on a DOPU image, the boundary of geographic atrophy while referring to an intensity image. That is, the signal processing unit 144 refers to the intensity image of the vicinity of the boundary of the binary image, and corrects the boundary of the binary image in accordance with the boundary position of the RPE layer in the intensity image. FIG. 7A illustrates the binary map 703 obtained by the binarization. The binary map 703, which is a binarized image, displays an atrophic defect (discrete region) in the RPE layer, an optic disk, blood vessels, and noise (not shown) in white. From among those displayed on the binary map 703, the signal processing unit 144 (identifying unit) detects (identifies) a defect in the RPE layer as a geographic atrophy region.

The present embodiment describes a method of manually selecting a geographic atrophy region. In step S602, the user selects a region. For this, the signal processing unit 144 displays a selected-region indicating circle 704 on the binary map 703 as in FIG. 7A. With the selected-region indicating circle 704, the user can specify any location and size. The two-dimensional image displayed here is not limited to that obtained by binarizing the En face map 702. For example, a pattern indicating an identified discrete region may be superimposed on a two-dimensional image (an En face map of an intensity image) obtained by projecting at least part of a three-dimensional tomographic intensity image onto a predetermined plane. As the at least part of the three-dimensional tomographic intensity image, for example, a region in the depth direction may be selected on the basis of a result of segmentation. The pattern indicating the identified discrete region is, for example, a line representing the range (contour) of the identified discrete region.

In step S603, the user determines whether the specified range is correct. If the range is correctly specified as the range of geographic atrophy, the signal processing unit 144 changes the color of the binarized portion within a geographic atrophy region 706, as illustrated in FIG. 7B, to highlight the geographic atrophy region 706 in step S604, thereby indicating that a geographic atrophy has been identified. In step S605, the user determines whether to end the analysis. If there are a plurality of geographic atrophy regions 706, the process returns to step S602, where the user can select a geographic atrophy region again. If the analysis concludes that there are a plurality of geographic atrophy regions 706, the signal processing unit 144 identifies each of them as a geographic atrophy and assigns numbers to them. Additionally, the signal processing unit 144 calculates the area of each of the geographic atrophy regions 706. Besides the areas, the barycentric coordinates of the geographic atrophy regions 706 may also be calculated.

If the user determines that the geographic atrophy region 706 has been correctly selected, the user can terminate the analysis with the button 705 illustrated in FIG. 7A and return to the display screen 501 illustrated in FIG. 5. In this case, the images displayed by the signal processing unit 144 are not limited to the En face map of the intensity image and the DOPU image. The signal processing unit 144 may display the DOPU image and the intensity image, or the En face map of the DOPU image and the intensity image. For example, the En face map of the intensity image and the binary map may he displayed. In this case, the location of the atrophic region in the RPE layer can be viewed on the En face map of the intensity image. For example, displaying the numbered geographic atrophy regions 706 on the En face map can facilitate viewing of atrophic regions in the RPE layer. Also, controlling the image density using a slider, with a plurality of images or maps superimposed on each other, can facilitate viewing of the location of a lesion.

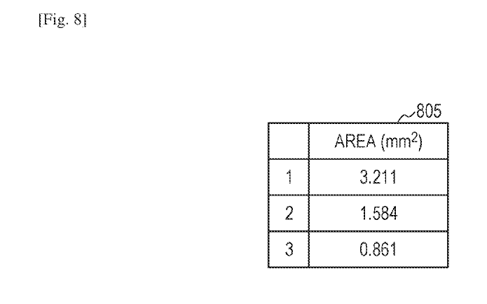

A list 805 of regions (see FIG. 8), each identified as a geographic atrophy, may be displayed in the list display section 522 of the display screen 501 (see FIG. 5). The regions in the list 805 are preferably generated in descending order of geographic atrophy area. This is because a geographic atrophy with a larger area is more likely to be diagnostically important. Analyzed information can be displayed together with the list 805. Although areas are displayed in the present embodiment because geographic atrophy is a lesion showing atrophy in the RPE layer, the barycentric coordinates of geographic atrophies may be listed instead. Although geographic atrophy regions are manually detected in the present embodiment, they may be automatically detected in accordance with an algorithm for detecting geographic atrophy near the central fovea. In the present embodiment described above, by accurately analyzing geographic atrophy using a DOPU image, the user can confirm the diagnosis, progression, and effect of treatment of atrophic age-related macular degeneration.

### Other Embodiments

Embodiments of the present invention can also be realized by a computer of a system or apparatus that reads out and executes computer executable instructions recorded on a storage medium (e.g., non-transitory computer-readable storage medium) to perform the functions of one or more of the above-described embodiment(s) of the present invention, and by a method performed by the computer of the system or apparatus by, for example, reading out and executing the computer executable instructions from the storage medium to perform the functions of one or more of the above-described embodiment(s). The computer may comprise one or more of a central processing unit (CPU), micro processing unit (MPU), or other circuitry, and may include a network of separate computers or separate computer processors. The computer executable instructions may be provided to the computer, for example, from a network or the storage medium. The storage medium may include, for example, one or more of a hard disk, a random-access memory (RAM), a read only memory (ROM), a storage of distributed computing systems, an optical disk (such as a compact disc (CD), digital versatile disc (DVD), or Blu-ray Disc (BD)™), a flash memory device, a memory card, and the like.

While the present invention has been described with reference to exemplary embodiments, it is to be understood that the invention is not limited to the disclosed exemplary embodiments. The scope of the following claims is to be accorded the broadest interpretation so as to encompass all such modifications and equivalent structures and functions.

This application claims the benefit of Japanese Patent Application No. 2015-001678 filed Jan. 7, 2015, No. 2015-001679 filed Jan. 7, 201.5, No. 2015-234268 filed Nov. 30, 2015, and No. 2015-234269 tiled Nov. 30, 2015, which are hereby incorporated by reference herein in their entirety,

1. An image processing apparatus comprising:
an extracting unit configured to extract a depolarizing region in a polarization-sensitive tomographic image of a subject's eye;
a detecting unit configured to detect, in a tomographic intensity image of the subject's eye, a region corresponding to the extracted depolarizing region, the tomographic intensity image corresponding to the polarization-sensitive tomographic image;
a calculating unit configured to calculate a size of the detected region; and
a display control unit configured to cause a display unit to display the detected region superimposed on the tomographic intensity image and to cause the display unit to display the calculated size in a state of associating the calculated size with the detected region.

2. The image processing apparatus according to claim 1, wherein the detecting unit detects, in the tomographic intensity image, a position of the region corresponding to the extracted depolarizing region by using information about a position of the extracted depolarizing region in the polarization-sensitive tomographic image,
wherein the detecting unit detects a contour of the region corresponding to the extracted depolarizing region by using information about the detected position and the tomographic intensity image.

3. The image processing apparatus according to claim 1, wherein the polarization-sensitive tomographic image and the tomographic intensity image are positionally associated with each other.

4. The image processing apparatus according to claim 1, further comprising a generating unit configured to split interference light of return light from the subject's eye irradiated with measuring light and reference light corresponding to the measuring light into a plurality of polarization components, and generate the polarization-sensitive tomographic image and the tomographic intensity image using information about the plurality of polarization components.

5. The image processing apparatus according to claim 4, wherein the generating unit generates the polarization-sensitive tomographic image using information about an output from a tomographic imaging apparatus connected to the image processing apparatus to be able to communicate therewith.

6. The image processing apparatus according to claim 1, wherein the polarization-sensitive tomographic image is a degree of polarization uniformity image.

7. The image processing apparatus according to claim 1, wherein the depolarizing region includes a retinal pigment epithelium layer and a lesion area; and
the detecting unit detects a region corresponding to the lesion area in the tomographic intensity image.

8. The image processing apparatus according to claim 1, wherein the display control unit causes the display unit to display the detected region superimposed on the tomographic intensity image in a color different from that of the tomographic intensity image.

9. (canceled)

10. (canceled)

11. An image processing apparatus comprising:
an extracting unit configured to extract a depolarizing region in a polarization-sensitive tomographic image of a subject's eye;
a detecting unit configured to detect, in a tomographic intensity image of the subject's eye, a region corresponding to the extracted depolarizing region, the tomographic intensity image corresponding to the polarization-sensitive tomographic image; and
a calculating unit configured to calculate a size of the detected region.

12. An image processing apparatus comprising:
an extracting unit configured to extract a depolarizing region in a three-dimensional polarization-sensitive tomographic image of a subject's eye;
a generating unit configured to generate a two-dimensional image obtained by projecting the extracted depolarizing region onto a predetermined plane; and
an identifying unit configured to identify at least one discrete region in the generated two-dimensional image.

13. An image processing apparatus comprising:
an extracting unit configured to extract a depolarizing region in a three-dimensional polarization-sensitive tomographic image of a subject's eye;
a detecting unit configured to detect, in a three-dimensional tomographic intensity image of the subject's eye, a region corresponding to the extracted depolarizing region, the three-dimensional tomographic intensity image corresponding to the three-dimensional polarization-sensitive tomographic image;
a generating unit configured to generate a two-dimensional image obtained by projecting the detected region onto a predetermined plane; and
an identifying unit configured to identify at least one discrete region in the generated two-dimensional image.

14. An image processing method comprising:
an extracting step of extracting a depolarizing region in a polarization-sensitive tomographic image of a subject's eye;
a detecting step of detecting, in a tomographic intensity image of the subject's eye, a region corresponding to the extracted depolarizing region, the tomographic intensity image corresponding to the polarization-sensitive tomographic image;
a calculating step of calculating a size of the detected region; and
a display step of causing a display unit to display the detected region superimposed on the tomographic intensity image and causing the display unit to display the calculated size in a state of associating the calculated size with the detected region.

15. An image processing method comprising:
an extracting step of extracting a depolarizing region in a polarization-sensitive tomographic image of a subject's eye;
a detecting step of detecting, in a tomographic intensity image of the subject's eye, a region corresponding to the extracted depolarizing region, the tomographic intensity image corresponding to the polarization-sensitive tomographic image; and
a calculating step of calculating a size of the detected region.

16. An image processing method comprising:
an extracting step of extracting a depolarizing region in a three-dimensional polarization-sensitive tomographic image of a subject's eye;
a detecting step of detecting, in a three-dimensional tomographic intensity image of the subject's eye, a region corresponding to the extracted depolarizing region, the three-dimensional tomographic intensity image corresponding to the three-dimensional polarization-sensitive tomographic image;
a generating step of generating a two-dimensional image obtained by projecting the detected region onto a predetermined plane; and
an identifying step of identifying at least one discrete region in the generated two-dimensional image.

17. An image processing method comprising:
an extracting step of extracting a depolarizing region in a three-dimensional polarization-sensitive tomographic image of a subject's eye;
a generating step of generating a two-dimensional image obtained by projecting the detected depolarizing region onto a predetermined plane; and
an identifying step of identifying at least one discrete region in the generated two-dimensional image.

18. A non-transitory computer-readable storage medium storing a program for causing a computer to execute each step of the image processing method according to claim 14.
